(19) United States
(12) Patent Application Publication
(10) Pub. No.: US 2024/0108870 A1
(43) Pub. Date: Apr. 4, 2024

(54) IMPLANTABLE THERAPEUTIC SUBSTANCE DELIVERY

(71) Applicant: Cochlear Limited, Macquarie University, NSW (AU)

(72) Inventors: Jonathon KIRK, Sydney (AU); Daniel SMYTH, Dublin (IE); Wolfram Frederik DUECK, Berlin (DE)

(21) Appl. No.: 18/553,792

(22) PCT Filed: Mar. 18, 2022
(86) PCT No.: PCT/IB2022/052495

§ 371 Date: Oct. 3, 2023

Related U.S. Application Data
(60) Provisional application No. 63/175,112, filed on Apr. 15, 2021.

(51) Int. Cl. A61M 31/00; A61F 11/00

(52) U.S. Cl. CPC A61M 31/002; A61F 11/00; A61M 2210/0662

(57) ABSTRACT

Presented herein are devices configured to deliver therapeutic substances to a fluidically-sealed chamber within the body of a recipient. The devices presented herein comprise a first/primary layer or substrate configured to be positioned adjacent a tissue barrier associated with the fluidically-sealed chamber. An adhesive is disposed on the primary layer to adhere the primary layer to the tissue barrier and provide a fluidic seal between the primary layer and the tissue barrier. In addition, a plurality of bioresorbable protrusions extend from the first layer and are configured to penetrate the tissue barrier and deliver a therapeutic substance to the fluidically-sealed chamber.

### BACKGROUND

### Field of the Invention

The present invention relates generally to the implantable delivery of therapeutic substances to a recipient.

### Related Art

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In one aspect, an apparatus is provided. The apparatus comprises: a primary layer comprising a first surface configured to be positioned abutting a tissue barrier associated with a fluidically-sealed chamber within a body of a recipient; a plurality of bioresorbable protrusions extending from the first surface and configured to form openings in the tissue barrier and deliver one or more therapeutic substances to the fluidically-sealed chamber; and an adhesive disposed on at least a portion of the first surface configured to adhere the first surface to the tissue barrier and fluidically-seal the openings formed by the plurality of bioresorbable protrusions.

In another aspect, a method is provided. The method comprises: accessing a tissue barrier associated with a fluidically-sealed chamber in a body of a recipient; positioning a self-sealing delivery device adjacent the tissue barrier, wherein the self-sealing delivery device comprises a primary layer and a plurality of bioresorbable protrusions extending from the primary layer; inserting the plurality of bioresorbable protrusions through the tissue barrier; fluidically-sealing the tissue barrier with the primary layer; and delivering one or more therapeutic substances to the fluidically-sealed chamber via the self-sealing delivery device.

In another aspect, an apparatus is provided. The apparatus comprises: a bioresorbable substrate configured to be adhered to a proximal surface tissue barrier in a body of a recipient; and a plurality of bioresorbable protrusions extending from the bioresorbable substrate, wherein the plurality of bioresorbable protrusions have a longitudinal rigidity to open perforations in the tissue barrier, and wherein the bioresorbable substrate is configured to fluidically seal the perforations opened in the tissue barrier.

In another aspect, an apparatus is provided. The apparatus comprises: a primary layer having a first surface configured to be positioned abutting a tissue barrier associated with a fluidically-sealed chamber within a body of a recipient and a second surface disposed opposite the first surface; a plurality of bioresorbable protrusions extending from the first surface and configured to form openings in the tissue barrier and deliver one or more therapeutic substances to the fluidically-sealed chamber, wherein the plurality of bioresorbable protrusions have a variable spacing there between with at least a minimum spacing; an adhesive disposed on at least a portion of the first surface configured to adhere the first surface to the tissue barrier and fluidically-seal the openings formed by the plurality of bioresorbable protrusions, wherein the primary layer is from a resiliently flexible and bioresorbable material configured to conform to a shape of a proximal surface of the tissue barrier, wherein one or more of the plurality of bioresorbable protrusions each include, or are coated by, at least one of the one or more therapeutic substances, and wherein the primary layer includes at least one therapeutic sub stance.

### BRIEF DESCRIPTION OF THE DRAWINGS

Embodiments of the present invention are described herein in conjunction with the accompanying drawings, in which:

FIG. 1 is a schematic diagram illustrating the ear of a recipient and a self-sealing therapeutic substance delivery device, in accordance with certain embodiments presented herein.

FIG. 2A is a cross-sectional view of a self-sealing therapeutic substance delivery device, in accordance with certain embodiments presented herein;

FIG. 2B is a top view of the self-sealing therapeutic substance delivery device of FIG. 2A;

FIG. 3A is a top view of a self-sealing therapeutic substance delivery device, in accordance with certain embodiments presented herein;

FIG. 3B is a top view of another self-sealing therapeutic substance delivery device, in accordance with certain embodiments presented herein;

FIG. 3C is a top view of another self-sealing therapeutic substance delivery device, in accordance with certain embodiments presented herein;

FIG. 5 is a cross-sectional view of another self-sealing therapeutic substance delivery device, in accordance with certain embodiments presented herein;

FIG. 6 is a cross-sectional view of another self-sealing therapeutic substance delivery device, in accordance with certain embodiments presented herein;

FIG. 7A is a cross-sectional view of a self-sealing therapeutic substance delivery device, in accordance with certain embodiments presented herein;

FIG. 7B is a top view of the self-sealing therapeutic substance delivery device of FIG. 7A;

FIG. 8 is a cross-sectional view of another self-sealing therapeutic substance delivery device and an insertion device, in accordance with certain embodiments presented herein; and

FIG. 9 is a flowchart illustrating an example method, in accordance with certain embodiments presented herein.

### DETAILED DESCRIPTION

A growing area of research and development relates to the use of pharmaceutical compounds, biological substances, bioactive substances, etc., including pharmaceutical agents/active pharmaceutical ingredients (APIs), genes, messenger RNA (mRNA) or other signalling compounds that promote recovery and resolution, chemicals, ions, drugs, etc. to treat a variety of disorders within the body of individual patient/recipient. These various substances, which are collectively and generally referred to herein as “therapeutic substances,” are delivered to induce some therapeutic results/treatment within the body of the recipient. For example, therapeutic substances may be delivered to treat ear disorders (e.g., tinnitus, hearing loss, tinnitus, Meniere's disease, etc.), to treat infections post-surgery, to fight cancer cells, to treat neurodegenerative diseases, to treat infectious diseases, etc.

The body of an animal, including the body of a human recipient (“recipient”), includes a number of different fluidically-sealed chambers (e.g., cavities or enclosed areas in which bodily fluids are sealed). For example, sensitive tissues in the body of a recipient, such as the brain, the ear, the eye, etc. are protected from the normal circulation by fluidic tissue barriers. In particular, the brain is surrounded by the blood-brain barrier (BBB), the inner ear (including the cochlea and the vestibular system) are surrounded by the blood-labyrinth barrier (BLB), the eye retina is surrounded by the blood—ocular barrier (BOB), which includes the blood-aqueous barrier (BAB) and the blood-retinal barrier (BRB), and so on. Other tissue barriers, such as the round window, and/or the oval window, are also present in the body of a recipient and are two tissue barriers associated with a fluidically-sealed cochlea of a recipient.

As noted, it can be advantageous to deliver therapeutic substances to the body of a recipient. However, using conventional techniques, it is difficult to deliver therapeutic substances to fluidically-sealed chambers within the body of a recipient without compromising the near-term or long-term structural and functional integrity of the associated fluidic tissue barrier (e.g., without introducing openings in the barrier that all either allow the fluid within the chamber to leak out and/or openings in the barrier that allow toxins, bacteria, viruses or other components to enter into the chamber, immediately or in the future). A tissue barrier that allows fluid within the chamber to leak out, or that allow toxins, bacteria, viruses or other components to enter into the chamber (e.g., a barrier with a compromised structural and functional integrity) is sometimes referred to herein as suffering from a “barrier disorder,” which in turn may cause malfunction of the organ(s) which it is designed to protect. Moreover, fluidically-sealed chambers are often located at positions within the body that prove difficult for a surgeon to access.

Presented herein are devices configured to deliver therapeutic substances to a fluidically-sealed chamber within the body of a recipient. The devices presented herein, sometimes referred to as “self-sealing therapeutic substance delivery devices” or simply “self-sealing delivery devices,” comprise a first/primary layer or substrate configured to be positioned adjacent a tissue barrier associated with the fluidically-sealed chamber. An adhesive is disposed on the primary layer to adhere the primary layer to the tissue barrier and provide a fluidic seal between the primary layer and the tissue barrier. In addition, a plurality of bioresorbable protrusions extend from the first layer and are configured to penetrate the tissue barrier and deliver a therapeutic substance within the chamber. In certain embodiments, the primary layer and the adhesive are also bioresorbable.

Merely for ease of description, the self-sealing delivery devices presented herein will primarily be described with reference to the delivery of therapeutic substances to a specific fluidically-sealed chamber of a recipient, namely the cochlea of a recipient behind the round window. However, it is to be appreciated that the self-sealing delivery devices presented herein can be used to deliver therapeutic substances to other fluidically-sealed chambers within the body of a recipient behind other tissue barriers.

It is also to be appreciated that the self-sealing delivery devices presented herein can be used alone or in combination with a number of different types of implantable medical devices. For example, the techniques presented herein may be implemented with auditory prostheses, such as middle ear auditory prostheses, bone conduction devices, direct acoustic stimulators, electro-acoustic prostheses, auditory brain stimulators, cochlear implants, combinations or variations thereof, etc. The techniques presented herein may also be used with tinnitus therapy devices, vestibular devices (e.g., vestibular implants), visual devices (i.e., bionic eyes), sensors, pacemakers, drug delivery systems, defibrillators, functional electrical stimulation devices, catheters, seizure devices (e.g., devices for monitoring and/or treating epileptic events), sleep apnea devices, electroporation devices, etc.

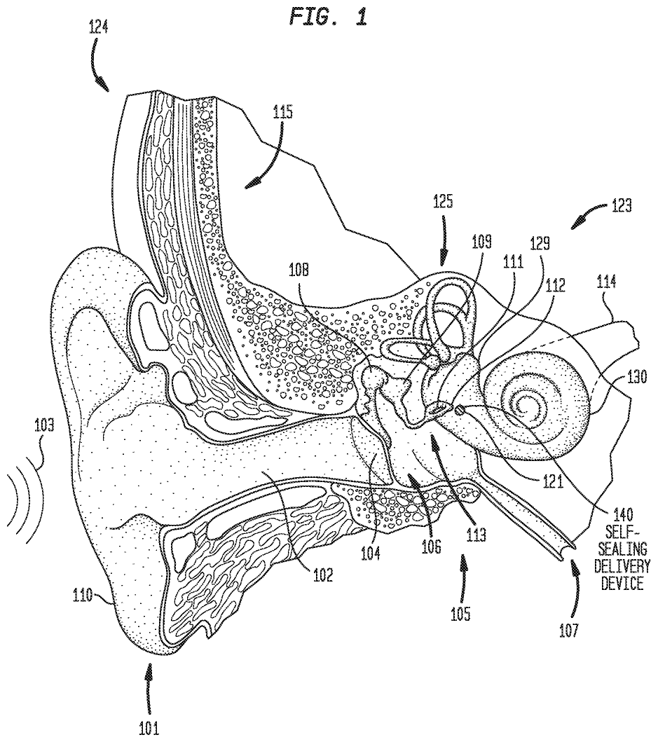

As noted, the self-sealing delivery devices are primarily described herein with reference to the delivery of therapeutic substances to the cochlea of a recipient. Before describing details of the self-sealing delivery devices, basic structures of the ear of a recipient, including a cochlea with which a self-sealing delivery device may be used, are first described below with reference to FIG. 1. FIG. 1 also illustrates an example self-sealing delivery device configured to deliver one or more therapeutic substances to the cochlea of the recipient.

FIG. 1 illustrates that a recipient's ear normally comprises an outer ear 101, a middle ear 105, and an inner ear 107. In a fully functional ear, outer ear 101 comprises an auricle 110 and an ear canal 102. An acoustic pressure or sound wave 103 is collected by auricle 110 and channeled into and through ear canal 102. Disposed across the distal end of ear canal 102 is a tympanic membrane 104 which vibrates in response to sound wave 103. This vibration is coupled to oval window or fenestra ovalis 112, which is adjacent round window 121, through the bones of the middle ear 105. The bones of the middle ear 105 comprise the malleus 108, the incus 109 and the stapes 111, collectively referred to as the ossicles 106. The ossicles 106 are positioned in the middle ear cavity 113 and serve to filter and amplify the sound wave 103, causing oval window 112 to articulate (vibrate) in response to the vibration of tympanic membrane 104. This vibration of the oval window 112 sets up waves of fluid motion of the perilymph within cochlea 130. Such fluid motion, in turn, activates tiny hair cells (not shown) inside of cochlea 130. Activation of the hair cells causes appropriate nerve impulses to be generated and transferred through the spiral ganglion cells (not shown) and auditory nerve 114 to the brain (also not shown) where they are perceived as sound

The human skull is formed from a number of different bones that support various anatomical features. Illustrated in FIG. 1 is the temporal bone 115 which is situated at the side and base of the recipient's skull 124 (covered by a portion of the recipient's skin/muscle/fat, collectively referred to herein as tissue 119). Also shown in FIG. 1 is the bony labyrinth 123, which is a rigid bony structure surrounding the inner ear 107. The bony labyrinth 123 consists of the semicircular canals 125, the vestibule 129, and the cochlea 130, which are chambers/cavities hollowed out of the substance of the bone, and lined by periosteum. The semicircular canals 125, the vestibule 129, and the cochlea 130 each include a corresponding portion of the membranous labyrinth (e.g., the vestibule contains the utricle and saccule, each semicircular canal contains a semicircular duct, and the cochlea contains the cochlear duct). The membranous labyrinth is filled with a fluid called endolymph, and surrounding the membranous labyrinth and filling the remaining space in the bony labyrinth 123 is the perilymph.

The bony labyrinth 123 includes two membrane-covered openings, the oval window 112 (oval window membrane) and the round window 121 (round window membrane). As noted, the oval window 112 vibrates in response to the vibration of tympanic membrane 104. The cochlea 130 is a closed, fluid-filled chamber such that the round window 121 vibrates with opposite phase to the vibrations entering the cochlea 130 through the oval window 112. As such, the round window 121 allows the perilymph in the cochlea 130 to move (in response to vibration at the oval window 112), which in turn ensures that hair cells of the basilar membrane will be stimulated and that audition will occur. The oval window 112 (oval window membrane) and the round window 121 (round window membrane) are tissue barriers that maintain the fluidic seal of the cochlea 130.

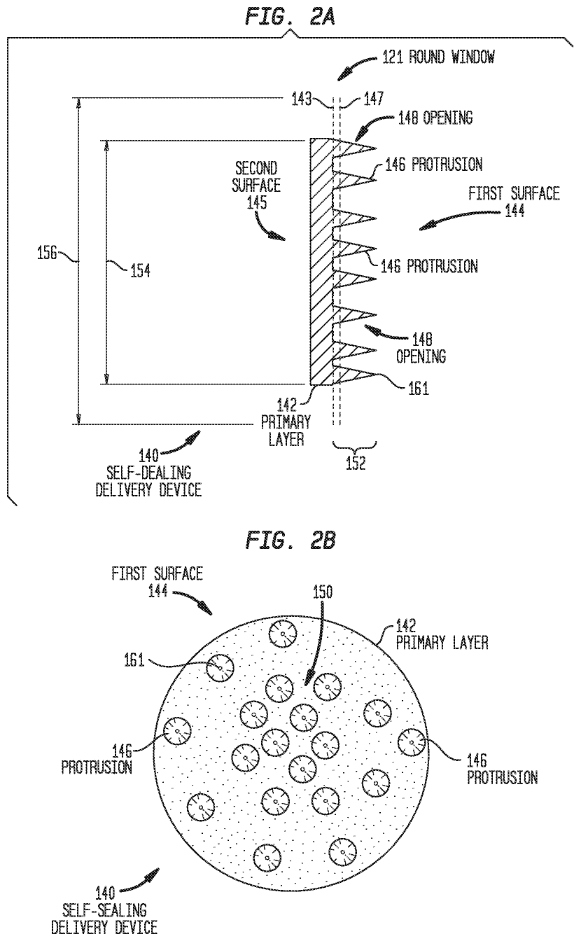

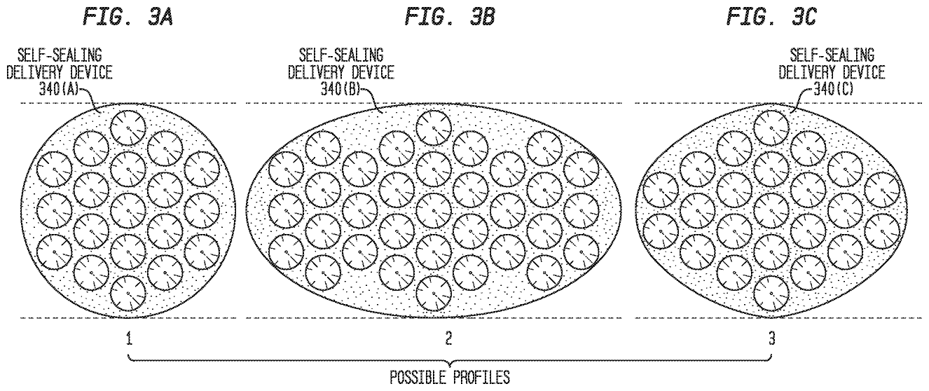

Since the cochlea 130 is a fluidically-sealed chamber, maintaining the fluidic seal thereof is important to, for example, maintain residual hearing in the cochlea 130, ensure the integrity of the blood-labyrinth barrier, etc. In addition, the cochlea 130 is located under the temporal bone 115 and is a small structure, thus it is difficult for a surgeon to access. However, despite these issues, it may be beneficial to deliver/introduce therapeutic substances into the cochlea 130. To this end, FIG. 1 illustrates a self-sealing therapeutic substance delivery device (self-sealing delivery device) 140 in accordance with embodiments presented herein. As shown, the self-sealing delivery device 140 is positioned on, and attached to, to an outside surface of the round window 121, but is configured to deliver therapeutic substances into the cochlea 130 (e.g., through the round window). FIGS. 2A and 2B are schematic diagrams illustrating further details of the self-sealing delivery device 140.

More specifically, FIG. 2A is a cross-sectional view of the self-sealing delivery device 140 and the round window 121, while FIG. 2B is top view of the self-sealing delivery device 140, shown separate from the round window 121. For ease of description, FIGS. 2A and 2B will generally be described together.

As shown, the self-sealing delivery device 140 comprises a first or primary layer/substrate 142 having a first surface 144 and a second surface 145, disposed opposite to the first surface. In use, the first surface 144 is configured to be positioned immediately adjacent to (e.g., abutting) an outside/proximal surface 143 of a tissue barrier which, in this example, is the round window 121. As such, in use, the second surface 145 is generally positioned facing away from the proximal surface 143 of the round window 121.

The self-sealing delivery device 140 further comprises a plurality of elongate protrusions 146 extending from the first surface 144 of the primary layer 142. The protrusions 146 each have a longitudinal rigidity such that, when the first surface 144 is placed adjacent to the proximal surface 143 of the round window 121, the protrusions 146 will penetrate the round window 121 (e.g., create perforations/openings 148 in the round window 121). That is, in the final implanted location shown in FIG. 2A, the first surface 144 is abutting the proximal surface 143 of the round window 121 (or other target tissue barrier) and the protrusions 146 extend through the openings 148 into the cochlea 130 (e.g., past a distal surface 147 of the round window 121). As a result, the protrusions 146 are located inside the cochlea 130.

In certain embodiments, the elongate protrusions 146 can include a variable elongate profile (e.g., width or dimensions that change along the elongate length) which could, for example, aid retention. For example, in one such embodiments, one or more of the protrusions could widen to a defined profile which then is reduced to a “neck,” such as a barb or comparable shape. This feature could work with multiple protrusions.

As noted above, the cochlea 130 is a fluid-filled (e.g., perilymph-filled) chamber and functions as closed-system where vibration of the oval window 112 causes (via fluid transfer) opposite vibration of the round window 121. Therefore, when the protrusions 146 extend through the round window 121, the self-sealing delivery device 140 is further configured to fluidically-seal the openings 148 in the round window 121. As such, in the example of FIGS. 2A and 2B, the primary layer 142 is resiliently flexible and includes an adhesive 150 disposed in or on the first surface 144. The adhesive 150 is configured to adhere the first surface 144 to the proximal surface 143 of the round window 121. Due to the flexible nature of the primary layer 142, the adhesive 150 is able to conform the primary layer 142 so that a fluidic seal is formed between the first surface 144 and the round window 121. As a result, the primary layer 142 prevents the fluid (e.g., perilymph) from leaking out of the cochlea 130 via the openings 148, and the self-sealing delivery device 140 blocks ingress to invasive pathogens via the openings 148. Stated differently, the primary layer 142 provides a two-way (bidirectional) seal to protect the cochlea 130 from fluid leakage and to block invasive pathogens from entering the cochlea 130.

In certain embodiments, the adhesive 150 comprises an adhesive gel or adhesive film disposed on the first surface 144. In other embodiments, the adhesive 150 is integrated into the first surface 144 (e.g., the first surface is formed so as to have adhesive properties). In such embodiments in which the e adhesive 150 is integrated into the first surface 144, a covering can be provided on the first surface 144 prior to implantation of the self-sealing delivery device 140 into a recipient. In any event, the adhesive 150 ensures that the self-sealing delivery device 140 remains abutting the round window 121 to seal the cochlea 130.

As noted above, a purpose of the self-sealing delivery device 140 is to deliver therapeutic substances to the cochlea 130. Therefore, in the example of FIGS. 2A and 2B, the protrusions 146 can be formed from, or include, one or more therapeutic substances to be delivered to the cochlea 130. In certain embodiments, one or more therapeutic substances are integrated into the protrusions 146 and are released into the cochlea 130 via, for example, elution, resorption, etc. For example, the protrusions 146 can be formed using a scaffold or crystalline structure where the scaffold/crystal structure is loaded/doped with one or more therapeutic substances. In other embodiments, the protrusions 146 can be coated with one or more therapeutic substances.

The protrusions 146 can be micro-machined, etched, or fabricated using deposition-based manufacturing methods or lithographic methods or through controlled-crystallization. The protrusions 146 generally have a length 152 that is sufficient to penetrate the round window 121, or other target tissue barrier. For example, the human round window has a thickness in the range of about 70 um+/−30 um, thus the protrusions 146 may have a length that is about at least twice this length or a length up to about seven (7) times (e.g., lengths of about 150 um to approximately 500 um length). However, it is also to be appreciated that the protrusions 146 can have different lengths in alternative embodiments.

It is also to be appreciated that the protrusions 146 can have similar or different arrangements to one another. For example, the protrusions 146 can have the same lengths or different lengths, the same lateral dimensions (e.g., diameter) or different lateral dimensions, the same therapeutic substances or different therapeutic substances, etc. In one illustrative arrangement, one or more protrusions 146 can include a first one or more therapeutic substances, while one or more other protrusions 146 can include a second one or more therapeutic substances that are different from the first one or more therapeutic substances. In such embodiments, the different therapeutic substances can have different therapeutic effects, different release profiles (e.g., different release timelines), and/or other differences. In a similar manner, a single protrusion 146 could include two or more different therapeutic substances having different therapeutic effects, different release profiles, and/or other differences. For example, by incorporating degrading protrusions which are coated in, or constructed of, different therapeutic substances, it is possible to produce a combination of burst and sustained release profiles.

As noted, the protrusions 146 are configured to form openings 148 in the round window 121. As such, the protrusions 146 can each include a distal end 161 that is configured to perforate the round window 121. In certain examples, the distal ends 161 can be configured to minimize or limit damage to the round window 121 when forming the openings 148. In certain embodiments, one or more of the distal ends 161 can have a conical shape with a distally facing point. The conical shape can comprise, for example, at least one of a nail point, a cone point, or a Type 17 point, a Type 23 point, etc. In certain embodiments, one or more of the protrusions 146 can include a threaded body configured be screwed through the membrane.

In certain embodiments, the adhesive 150 and/or the first surface 144 can also include one or more therapeutic substances for delivery to the round window 121. The one or more therapeutic substances for delivery to the round window 121 can be the same or different from the one or more therapeutic substances delivered into the cochlea 130. For example, the one or more therapeutic substances in or on the adhesive 150 and/or the first surface 144 can be specifically configured to promote healing of the openings 148 in the round window 121.

In certain embodiments, one or more portions of the adhesive 150 and/or the first surface 144 can include a first one or more therapeutic substances, while one or more other portions of the adhesive 150 and/or the first surface 144 can include a second one or more therapeutic substances that are different from the first one or more therapeutic substances. In such embodiments, the different therapeutic substances can have different therapeutic effects, different release profiles (e.g., different release timelines), and/or other differences. In a similar manner, a single portion of the adhesive 150 and/or the first surface 144 could include two or more different therapeutic substances having different therapeutic effects, different release profiles, and/or other differences.

In the examples of FIGS. 2A and 2B, the protrusions 146 are fully bioresorbable, meaning the protrusions 146 will, over time, be fully resorbed by the body (e.g., the protrusions are constructed of a degradable material which may or may not be the therapeutic compound or drug and serves as the scaffold and support for the therapeutic substance). In certain embodiments, the adhesive 150 and the primary layer 142 are also fully bioresorbable, meaning that the entire self-sealing delivery device 140 is bioresorbable. A fully/completely bioresorbable self-sealing delivery device 140 can be advantageous in that the round window 121 (or other tissue barrier) only needs to be accessed a single time during the initial implantation, and there is no need for a second surgical procedure to remove the self-sealing delivery device 140 after the therapeutic substance(s) have been delivered and/or to leave the surgical site open for an extended period (e.g., to allow time for delivery of the therapeutic substance and subsequent removal).

The self-sealing delivery device 140 can bioresorb via dissolution and/or hydrolysis. For example, if the self-sealing delivery device 140 has a salt structure or is in a form or have sufficient polar properties to be attracted to water molecules, then the self-sealing delivery device 140 will readily dissolve over time. In the case that the self-sealing delivery device 140 is a polymer, the self-sealing delivery device 140 can be synthetically tailored to include polar functional groups that allow water to break the polymer down to smaller form that accelerates degradation and biological clearance. Poly-lactic-co-glycolic acid, and polyvinyl alcohol are two example, tunable (i.e. rate of degradation can be adjusted), biodegradable polymers. Alternatively, the self-sealing delivery device 140 could be formed from a naturally occurring polymer such as collagen matrices, and other complex sugars, proteins. The self-sealing delivery device 140 could also be formed from some inorganic calcium carbonates. Enzymatic breakdown and digestion is another alternative where the self-sealing delivery device 140 is slowly attacked and digested by cells in the body's immune response.

In addition, the protrusions 146 have lateral dimensions (e.g., diameters) and a pitch (spacing) there between to ensure that the openings 148 in the round window 121 do not damage the structural integrity of the round window 121. That is, if the openings 148 are within a certain size, and have a certain spacing, the openings 148 will heal over, with no histological evidence of being perforated, once the protrusions 146 degrade. As such, in accordance with embodiments presented herein, the lateral dimensions of the protrusions 146 are sufficiently small so that the openings 148 will self-heal following resorption of the protrusions 146. Moreover, the pitch (spacing) of the protrusions 146 is selected to ensure that the long-term viability of the round window 121 is not compromised by the openings 148. For example, the protrusions 146 can have a minimum spacing there between to ensure the long-term viability of the round window 121. In certain embodiments, the spacing between protrusions 146 is at least equal to the diameter of the protrusions.

Again, as noted above, FIGS. 2A and 2B illustrate a specific use of the self-sealing delivery device 140 at the round window 121. In certain such embodiments, the primary layer 142 has an outside lateral dimension (e.g., a diameter) 154 that is smaller than an outside lateral dimension (e.g., a diameter) 156 of the round window 121. As a result, the first surface 144 can be positioned fully on the round window 121 to provide the fluidic seal. In one embodiment, the primary layer 142 (and thus the first surface 144) has a lateral dimension 154 that is less than 1.2 mm. In another embodiment, the primary layer 142 (and thus the first surface 144) has a lateral dimension 154 that is less than 1 mm. In another embodiment, the primary layer 142 (and thus the first surface 144) has a lateral dimension 154 that is less than 0.5 mm.

Although, as noted above, certain embodiments ensure that the primary layer 142 has an outside lateral dimension 154 that is smaller than an outside lateral dimension 156 of the round window 121, it is also to be appreciated that the primary layer 142 could have an outside lateral dimension 154 that is larger than an outside lateral dimension 156 of the round window. For example, the primary layer 142 could be sized such that an outer edge 158 of the first surface 144 adheres to the bony capsule around the round window 121. In such embodiments, the fluidic seal could between the portions of the first surface 144 surrounding the openings 148 and the round window) and/or formed between the bony capsule and the outer edge 158 of the first surface 144 (e.g., the fluid within the cochlea 130 could pass through the openings 148, but is sealed at the outer edge 158).

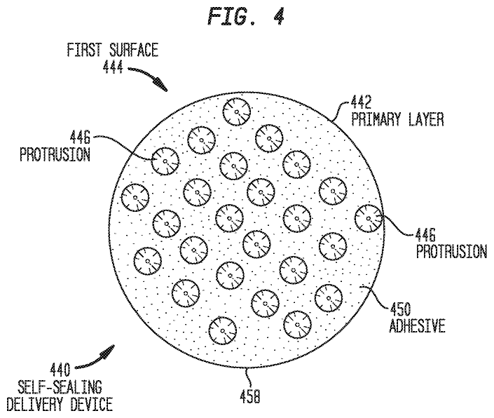

It is be appreciated that the specific configuration for the self-sealing delivery device 140 shown on FIGS. 2A and 2B is merely illustrative and that self-sealing delivery devices in accordance with embodiments presented herein can have a number of different configurations. For example, FIGS. 3A, 3B, and 3C illustrate three different profiles (lateral shapes) for self-sealing delivery devices in accordance with embodiments presented herein. In particular, FIG. 3A illustrates a self-sealing delivery device 340(A) having a generally circular profile, while FIG. 3B illustrates a self-sealing delivery device 340(B) having a generally elliptical profile. FIG. 3C illustrates a self-sealing delivery device 340(C) having a profile defined by two opposing parabolas with a same axis of symmetry. Again, these specific profiles shown in FIGS. 3A-3C are merely illustrative and other profiles can be used in alternative embodiments presented herein.

In addition to different profiles, self-sealing delivery devices presented herein can include different numbers of protrusions with the same or different protrusion pitch (spacing). In certain embodiments, self-sealing delivery devices presented herein can include a variable protrusion pitch. A variable protrusion pitch is a pitch that changes across the first surface of the primary layer. A variable protrusion pitch may be advantageous to, for example, to improve structural integrity of the round window or other tissue barrier, provide a higher density for improved therapeutic substance delivery, etc.

FIG. 4 is a schematic top view of an example self-sealing delivery device 440 having a variable protrusion pitch. Similar to the self-sealing delivery device 140 of FIGS. 2A and 2B, the self-sealing delivery device 440 comprises a primary layer 442 having a first surface 444 configured to be positioned abutting a tissue barrier. In addition, a plurality of protrusions 446 extend from the first surface 444, and an adhesive 450 is disposed in or on the first surface 444.

In addition, in the example of FIG. 4 the plurality of protrusions 446 have a variable pitch across the first surface 444. In particular, there is a higher density of protrusions 446 at a central region 457 of the first surface 444, but a lower density of protrusions 446 approaching an outer edge 458 of the first surface 444. Moreover, even at the central region 457, the protrusions 446 can have different spacing there between (e.g., with a minimum spacing to ensure integrity of the tissue barrier).

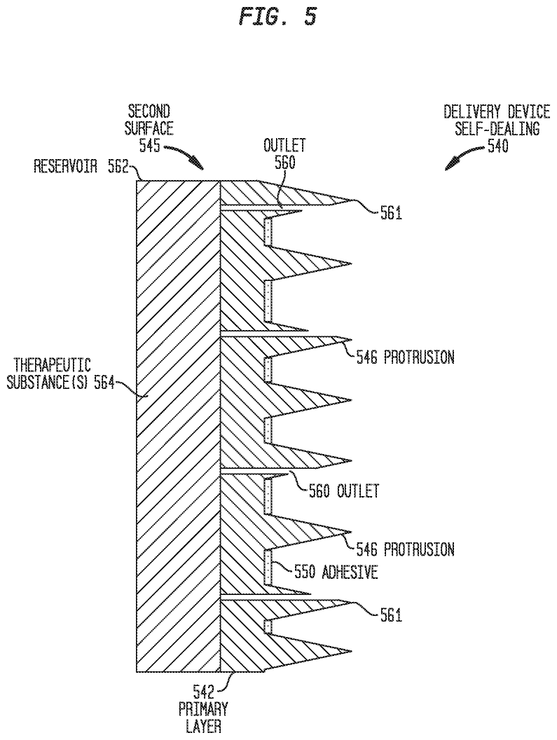

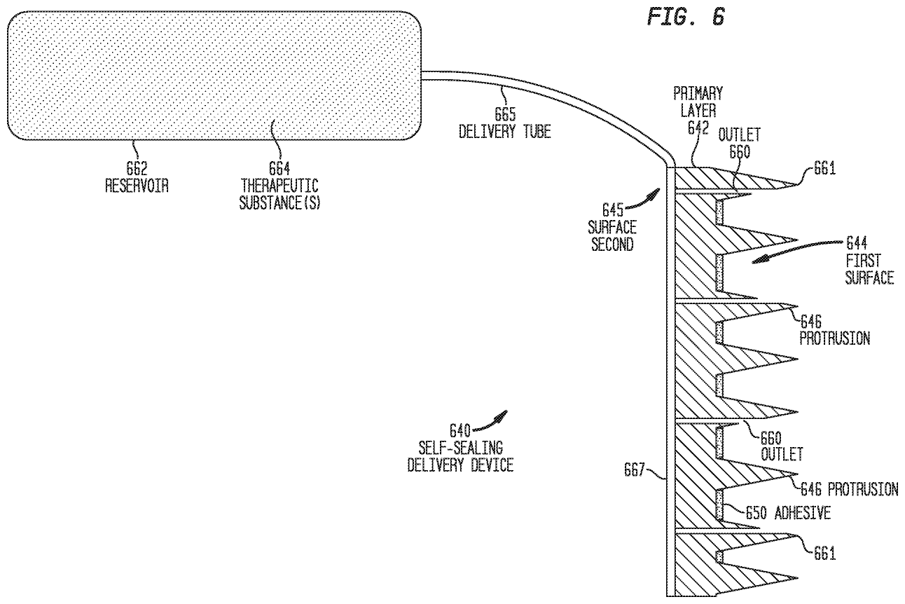

As noted, in certain embodiments presented herein, the protrusions are configured to penetrate a tissue barrier include one or more therapeutic substances. In other embodiments, one or more of the protrusions can also or alternatively function, at least temporarily, as a conduit for delivery of one or more therapeutic substances from a reservoir. FIGS. 5 and 6 illustrate examples of such embodiments.

More specifically, FIG. 5 is a cross-sectional view of an example self-sealing delivery device 540, in accordance with certain embodiments presented herein. Similar to the self-sealing delivery device 140 of FIGS. 2A and 2B, the self-sealing delivery device 540 comprises a primary layer 542 having a first surface 544 configured to be positioned abutting a tissue barrier. In addition, a plurality of protrusions 546 extend from the first surface 544, and an adhesive 550 is disposed in or on the first surface 544.

In addition, in the example of FIG. 5, one or more of the plurality of protrusions 546 include a through-hole or outlet 560 fluidically coupled to a reservoir 562 that is disposed in, or attached to, the second surface 545 of the primary layer 542. The reservoir 562 has one or more therapeutic substances 564 contained therein. In certain embodiments, the reservoir 562 can be bioresorbable.

In the embodiment of FIG. 5, once the protrusions 546 penetrate the tissue barrier, the outlets 560 enable the one or more therapeutic substances 564 contained in the reservoir 562 to enter the fluidically-sealed chamber behind the tissue barrier to which the self-sealing delivery device 540 is attached. The outlets 560 can be offset from a distal end 561 of the protrusions 546 (as shown in FIG. 5) or the outlets 560 can be aligned with the distal end 561 of the protrusions 546.

Similar to the above embodiments, the protrusions 546 can also include (e.g., have disposed therein or thereon) one or more therapeutic substances for delivery to the fluidically-sealed chamber behind the tissue barrier to which the self-sealing delivery device 540 is attached. The one or more therapeutic substances included in/on the protrusions 546 can be the same or different from the one or more therapeutic substances 564 contained in the reservoir 562.

FIG. 6 is a cross-sectional view of an example self-sealing delivery device 640, in accordance with certain embodiments presented herein. Similar to the self-sealing delivery device 140 of FIGS. 2A and 2B, the self-sealing delivery device 640 comprises a primary layer 642 having a first surface 644 configured to be positioned abutting a tissue barrier. In addition, a plurality of protrusions 646 extend from the first surface 644, and an adhesive 650 is disposed in or on the first surface 644.

In the example of FIG. 6, one or more of the plurality of protrusions 646 include a through-hole or outlet 660 fluidically coupled to a reservoir 662 that is physically separate from the primary layer. The reservoir 662 has one or more therapeutic substances 664 contained therein and is fluidically coupled to protrusions 646 via a delivery tube 665 and a closed channel 667 located at the second surface 645 of the primary layer 642. In certain embodiments, the reservoir 662 can be bioresorbable.

In the embodiment of FIG. 6, once the protrusions 646 penetrate the tissue barrier, the outlets 660 enable the one or more therapeutic substances 664 contained in the reservoir 662 to enter the fluidically-sealed chamber behind the tissue barrier to which the self-sealing delivery device 640 is attached. The outlets 660 can be offset from a distal end 661 of the protrusions 646 (as shown in FIG. 6) or the outlets 660 can be aligned with the distal end 661 of the protrusions 646.

Similar to the above embodiments, the protrusions 646 can also include (e.g., have disposed therein or thereon) one or more therapeutic substances for delivery to the fluidically-sealed chamber behind the tissue barrier to which the self-sealing delivery device 640 is attached. The one or more therapeutic substances included in/on the protrusions 646 can be the same or different from the one or more therapeutic substances 664 contained in the reservoir 662.

FIGS. 5 and 6 illustrate use of reservoirs to deliver therapeutic substances. In the same or other embodiments, the reservoir may be empty (e.g. initially or after delivery) and used to wick fluid from the target site into the reservoir for analysis and diagnostics (e.g., via capillary forces, but other modes are possible

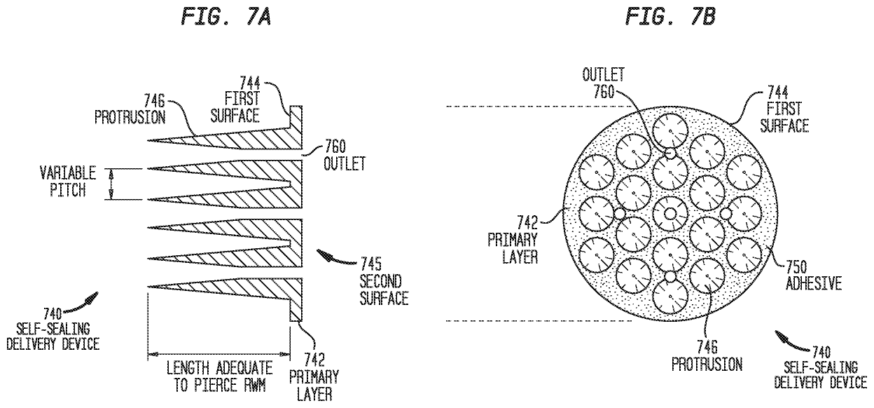

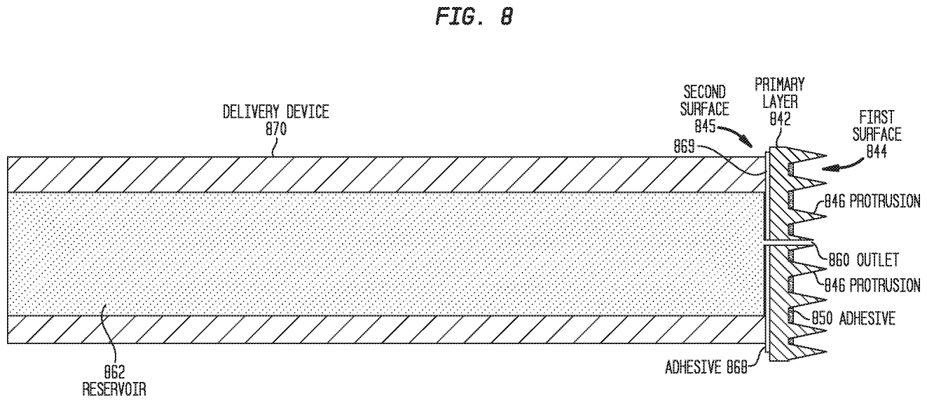

As noted, FIGS. 5 and 6 illustrate embodiments in which one or more of the protrusions can also or alternatively function, at least temporarily, as a conduit for delivery of one or more therapeutic substances from a reservoir. FIGS. 7A and 7B illustrate an alternative embodiment in which the first surface of a self-sealing delivery device is fluidically coupled to a reservoir for delivery of one or more therapeutic substances.

More specifically, FIG. 7A is a cross-sectional view of an example self-sealing delivery device 740, in accordance with certain embodiments presented herein, while FIG. 7B is a schematic top view of the self-sealing delivery device 740. For ease of description, FIGS. 7A and 7B will be described together.

Similar to the self-sealing delivery device 140 of FIGS. 2A and 2B, the self-sealing delivery device 740 comprises a primary layer 742 having a first surface 744 configured to be positioned abutting a tissue barrier. In addition, a plurality of protrusions 746 extend from the first surface 744, and an adhesive 750 is disposed in or on the first surface 744. In addition, in the example of FIGS. 7A and 7B, the primary layer 742 includes one or more through-holes or outlets 760 fluidically coupled to a reservoir (not shown in FIG. 7). The reservoir can disposed in, or attached to, the second surface 745 of the primary layer 742 (as in FIG. 5) or the reservoir can be fluidically coupled to outlets 760 via a closed channel located at the second surface 745 of the primary layer 742 (as in FIG. 6). The reservoir has one or more therapeutic substances 764 contained therein.

In the embodiment of FIGS. 7A and 7B, once the self-sealing delivery device 740 is positioned abutting the tissue barrier, the outlets 760 enable the one or more therapeutic substances contained in the reservoir to reach the tissue barrier. In certain embodiments, the outlets 760 can partially overlap with openings formed in the tissue barrier by the protrusions 746. In such embodiments, this overlap allows the one or more therapeutic substances delivered from the reservoir to enter the fluidically-sealed chamber behind the tissue barrier to which the self-sealing delivery device 740 is attached.

Similar to the above embodiments, the protrusions 746 can also include (e.g., have disposed therein or thereon) one or more therapeutic substances for delivery to the fluidically-sealed chamber behind the tissue barrier to which the self-sealing delivery device 740 is attached. The one or more therapeutic substances included in/on the protrusions 746 can be the same or different from the one or more therapeutic substances contained in the reservoir.

As noted above, self-sealing delivery devices in accordance with embodiments presented herein are configured to be attached (e.g., adhered) to a tissue barrier within the body of a recipient. As such, the self-sealing delivery devices needs to be positioned adjacent to the target tissue barrier. A variety of different techniques can be used to position a self-sealing delivery device in accordance with embodiments presented adjacent to the target tissue barrier, including techniques that do or do not make use of surgical devices/instruments.

In certain embodiments, a self-sealing delivery device in accordance with embodiments presented herein is configured to be configured as a detachable tip of a positioning/insertion instrument, such as a syringe. For example, FIG. 8 is a cross-sectional view of an example self-sealing delivery device 840, in accordance with certain such embodiments presented herein. Similar to the self-sealing delivery device 140 of FIGS. 2A and 2B, the self-sealing delivery device 840 comprises a primary layer 842 having a first surface 844 configured to be positioned abutting a tissue barrier and a second surface 845. In addition, a plurality of protrusions 846 extend from the first surface 844, and an adhesive 850 is disposed in or on the first surface 844.

FIG. 8 illustrates that the second surface 845 includes an attachment mechanism 868 configured to detachably mechanically couple attach the self-sealing delivery device 840 to the distal end 869 of an insertion instrument 870. In the specific example of FIG. 8, the attachment mechanism 868 is an adhesive that enables the self-sealing delivery device 840 to disengage from the distal end 869 of the insertion instrument 870, while the self-sealing delivery device 840 remains attached to the tissue barrier. For example, the adhesive 868 could have an adhesion strength that is less than the adhesion strength of adhesive 850, be an adhesive that is configured to rapidly dissolve or lose adhesion abilities when inserted into the body (e.g., via a temperature induced change, via a bodily fluid induces change, etc.), or other type of adhesive that enables the self-sealing delivery device 840 to disengage from the insertion instrument 870.

In certain embodiments, the insertion instrument 870 (e.g., syringe) could include a reservoir 862 with one or more therapeutic substances 864 contained therein. The self-sealing delivery device 840 could include one or more outlets 860, similar such as the outlets 560 or 660 shown in FIGS. 5 and 6, respectively, configured to deliver the one or more therapeutic substances 864 through the tissue barrier. For example, in the embodiment of FIG. 8, once the protrusions 846 penetrate the tissue barrier, the one or more outlets 860 enable the one or more therapeutic substances 864 contained in the reservoir 862 to enter the fluidically-sealed chamber behind the tissue barrier to which the self-sealing delivery device 840 is attached. The outlets 860 can be offset from a distal end of the protrusions 846 or the outlets 860 can be aligned with the distal end of the protrusions 846.

As noted, FIG. 8 illustrates the use of an adhesive to attach a self-sealing delivery device to an insertion device. It is to be appreciated that the use of an adhesive is merely illustrative and that other types of mechanical connectors can be used in alternative embodiments.

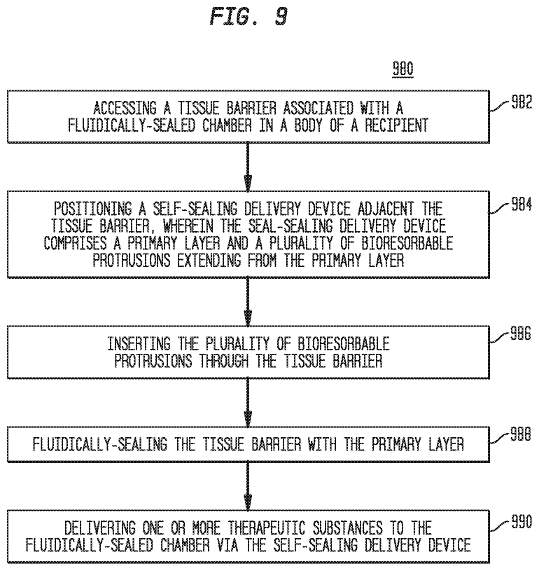

FIG. 9 is flowchart illustrating an example method 980, in accordance with embodiments presented herein. Method 980 begins at 982 where a surgeon accesses a tissue barrier associated with a fluidically-sealed chamber in a body of a recipient (e.g., forms a surgical opening in the body of the recipient and an access pathway to the tissue barrier). At 984, the surgeon positions a self-sealing delivery device adjacent the tissue barrier, wherein the self-sealing delivery device comprises a primary layer and a plurality of bioresorbable protrusions extending from the primary layer. At 986, the surgeon inserts the plurality of bioresorbable protrusions through the tissue barrier and, at 988, the primary layer fluidically-seals the tissue barrier. At 990, the self-sealing delivery device delivers one or more therapeutic substances to the fluidically-sealed chamber.

As should be appreciated, while particular uses of the technology have been illustrated and discussed above, the disclosed technology can be used with a variety of devices in accordance with many examples of the technology. The above discussion is not meant to suggest that the disclosed technology is only suitable for implementation within systems akin to that illustrated in the figures. In general, additional configurations can be used to practice the processes and systems herein and/or some aspects described can be excluded without departing from the processes and systems disclosed herein.

This disclosure described some aspects of the present technology with reference to the accompanying drawings, in which only some of the possible aspects were shown. Other aspects can, however, be embodied in many different forms and should not be construed as limited to the aspects set forth herein. Rather, these aspects were provided so that this disclosure was thorough and complete and fully conveyed the scope of the possible aspects to those skilled in the art.

As should be appreciated, the various aspects (e.g., portions, components, etc.) described with respect to the figures herein are not intended to limit the systems and processes to the particular aspects described. Accordingly, additional configurations can be used to practice the methods and systems herein and/or some aspects described can be excluded without departing from the methods and systems disclosed herein.

Similarly, where steps of a process are disclosed, those steps are described for purposes of illustrating the present methods and systems and are not intended to limit the disclosure to a particular sequence of steps. For example, the steps can be performed in differing order, two or more steps can be performed concurrently, additional steps can be performed, and disclosed steps can be excluded without departing from the present disclosure. Further, the disclosed processes can be repeated.

Although specific aspects were described herein, the scope of the technology is not limited to those specific aspects. One skilled in the art will recognize other aspects or improvements that are within the scope of the present technology. Therefore, the specific structure, acts, or media are disclosed only as illustrative aspects. The scope of the technology is defined by the following claims and any equivalents therein.

It is also to be appreciated that the embodiments presented herein are not mutually exclusive and that the various embodiments may be combined with another in any of a number of different manners. For example, the embodiments of FIG. 1 could be combined with any of the embodiments of FIG. 2A, 2B, 3A, 3B, 3C, 4, 5, 6, 7A, 7B, 8, or 9. Similarly, the embodiments of FIGS. 2A and 2B could be combined with any of the embodiments of FIG. 3A, 3B, 3C, 4, 5, 6, 7A, 7B, 8, or 9. Similarly, the embodiments of FIGS. 3A, 3B, and/or 3C could be combined with any of the embodiments of FIG. 4, 5, 6, 7A, 7B, 8, or 9. Similarly, the embodiments of FIG. 4 could be combined with any of the embodiments of FIG. 5, 6, 7A, 7B, 8, or 9. Similarly, the embodiments of FIG. 5 could be combined with any of the embodiments of FIG. 6, 7A, 7B, 8, or 9. Similarly, the embodiments of FIG. 6 could be combined with any of the embodiments of FIG. 7A, 7B, 8, or 9. Similarly, the embodiments of FIG. 7A or 7B could be combined with any of the embodiments of FIG. 8 or 9. Similarly, the embodiments of FIG. 8 could be combined with the embodiments of FIG. 9.

1. An apparatus, comprising:
a primary layer comprising a first surface configured to be positioned abutting a tissue barrier associated with a fluidically-sealed chamber within a body of a recipient;
a plurality of bioresorbable protrusions extending from the first surface and configured to form openings in the tissue barrier and deliver one or more therapeutic substances to the fluidically-sealed chamber; and
an adhesive disposed on at least a portion of the first surface configured to adhere the first surface to the tissue barrier and fluidically-seal the openings formed by the plurality of bioresorbable protrusions.

2. The apparatus of claim 1, wherein the primary layer and the adhesive are bioresorbable.

3. The apparatus of claim 1, wherein the adhesive comprises at least one of an adhesive gel disposed on the first surface, an adhesive film disposed on the first surface, or an adhesive integrated into the first surface.

4. (canceled)

5. (canceled)

6. The apparatus of claim 1, wherein the primary layer is resiliently flexible to conform to a shape of a proximal surface of the tissue barrier.

7. (canceled)

8. (canceled)

9. The apparatus of claim 8, wherein the one or more of the plurality of bioresorbable protrusions comprise a bioresorbable scaffold structure loaded with the at least one of the one or more therapeutic substances.

10. (canceled)

11. The apparatus of claim 7, wherein one or more of the plurality of bioresorbable protrusions are coated with at least one of the one or more therapeutic substances.

12. (canceled)

13. (canceled)

14. (canceled)

15. The apparatus of claim 1, wherein the one or more therapeutic substances comprise a plurality of therapeutic substances, and wherein a first one of the plurality of therapeutic substances has a first release profile, a second one of the plurality of therapeutic substances has a second release profile, and wherein the second release profile that is different from the first release profile.

16. The apparatus of claim 1, wherein one or more of the adhesive or the primary later includes at least one therapeutic substance.

17. (canceled)

18. (canceled)

19. (canceled)

20. (canceled)

21. The apparatus of claim 1, wherein the primary layer has an outer lateral dimension that is less than 1 mm.

22. The apparatus of claim 1, wherein the primary layer comprises a second surface that is generally opposite the first surface, and wherein the apparatus comprises:
at least one attachment mechanism disposed at the second surface, wherein the at least one attachment mechanism is configured to detachably mechanically couple the primary layer to an insertion instrument.

23. (canceled)

24. (canceled)

25. (canceled)

26. (canceled)

27. The apparatus of claim 1, further comprising:
a reservoir, wherein the reservoir contains at least one of the one or more therapeutic substances, and wherein one or more plurality of bioresorbable protrusions include one or more outlets that are fluidically-coupled to the reservoir.

28. (canceled)

29. (canceled)

30. The apparatus of claim 1, wherein one or more of the plurality of bioresorbable protrusions comprise a threaded body configured be screwed through the tissue barrier.

31. (canceled)

32. A method, comprising:
accessing a tissue barrier associated with a fluidically-sealed chamber in a body of a recipient;
positioning a self-sealing delivery device adjacent the tissue barrier, wherein the self-sealing delivery device comprises a primary layer and a plurality of bioresorbable protrusions extending from the primary layer;
inserting the plurality of bioresorbable protrusions through the tissue barrier;
fluidically-sealing the tissue barrier with the primary layer; and
delivering one or more therapeutic substances to the fluidically-sealed chamber via the self-sealing delivery device.

33. The method of claim 32, wherein the tissue barrier is a round window of a cochlea of the recipient, and wherein inserting the plurality of bioresorbable protrusions through the tissue barrier comprises:
inserting the plurality of bioresorbable protrusions through the round window.

34. The method of claim 32, wherein fluidically-sealing the tissue barrier with the primary layer comprises:
positioning the primary layer abutting a proximal surface of the tissue barrier; and
adhering the primary layer to the proximal surface of the tissue barrier.

35. The method of claim 34, wherein adhering the primary layer to the proximal surface of the tissue barrier comprises:
adhering the primary layer to the proximal surface via at least one of an adhesive gel disposed on a first surface of the primary layer or an adhesive film disposed on a first surface of the primary layer.

36. (canceled)

37. (canceled)

38. (canceled)

39. (canceled)

40. The method of claim 32, wherein delivering one or more therapeutic substances to the fluidically-sealed chamber via the self-sealing delivery device comprises:
delivering one or more therapeutic substances from a reservoir that is fluidically coupled to one or more outlets within the primary layer.

41. The method of claim 40, wherein the reservoir is integrated in the primary layer.

42. The method of claim 40, wherein the reservoir is physically separate from the primary layer and is fluidically-coupled to the one or more outlets via a delivery tube and the primary layer.

43-56. (canceled)
